(19) United States
(12) Patent Application Publication
(10) Pub. No.: US 2003/0053956 A1
(43) Pub. Date: Mar. 20, 2003

(54) Alkylaryl polyether alcohol polymers for treatment and prophylaxis of snoring, sleep apnea, sudden infant death syndrome and for improvement of nasal breathing

(76) Inventor: Thomas Hofmann, Seattle, WA (US)

Correspondence Address: HANA VERNY, PETERS, VERNY, JONES & SCHMITT, LLP, SUITE 6, 385 SHERMAN AVENUE, PALO ALTO, CA, 94306, US

(21) Appl. No.: 10/056,524

(22) Filed: Jan. 23, 2002

Related U.S. Application Data
(60) Provisional application No. 60/264,166, filed on Jan. 24, 2001.

(51) Int. Cl. A61L 9/04

(52) U.S. Cl. 424/45

(57) ABSTRACT

A method and composition for treatment and prophylaxis of snoring, sleep apnea or sudden infant death syndrome and for improvement of nasal breathing in mammals by nasal and/or pharyngeal administration of tyloxapol or a related alkylaryl polyether alcohol polymer. A spray, liquid or solid composition comprising from about 0.01 to about 20% (w/v), equivalent to about 100 μg/ml to about 200 mg/ml, of tyloxapol or another alkylaryl polyether alcohol polymer alone or in admixture with pharmaceutically acceptable excipients and additives. The composition is administered as a spray, liquid, liquid drops, lozenges or powder suitable for nasal and/or pharyngeal application.

This application is based on and claims priority of the provisional application Ser. No. 60/264,166 filed on Jan. 24, 2001.

### BACKGROUND OF THE INVENTION

1. Field of the Invention

The current invention concerns a method and composition for treatment and prophylaxis of snoring, sleep apnea or sudden infant death syndrome and for improvement of nasal breathing in mammals by nasal and/or pharyngeal administration of tyloxapol or a related alkylaryl polyether alcohol polymer. In particular, the present invention provides a spray, liquid or solid composition comprising from about 0.01 to about 20% (w/v), equivalent to about 100 μg/ml to about 200 mg/ml, of tyloxapol or another selected alkylaryl polyether alcohol polymer alone, in combination, or in admixture with pharmaceutically acceptable excipients and additives. The composition is administered as a spray, liquid, liquid drops, lozenges or powder suitable for nasal and/or pharyngeal application.

2. Background of the Invention

Snoring and related sleep apnea are amongst the most troublesome sleeping impairments. Snoring is not only a nuisance for other people, but it has been shown, similarly to sleep apnea, to correlate with increased daytime sleepiness and decreased alertness and work performance.

As a consequence of snoring and sleep apnea, normal sleep rhythm is disturbed and oxygen saturation is decreased ensuing in following tiredness and decrease in alertness and performance. Sleep apnea is characterized by repetitive episodes of upper airway obstruction that occurs during sleep and is usually associated with blood oxygen desaturation, snoring and daytime sleepiness.

Sleep apnea is defined as cessation of air flow for more than ten seconds, occurring at least ten times per hour at night (Clinics in Chest Medicine, 19:1 (1998) and Diagnostic and Coding Manual, The International Classification System of Sleep Disorders, Rochester, Minn. (1990)).

Sleep apnea often leads to increased blood pressure, EKG changes, arrhythmia, neurologic changes, and even to increased risk for stroke (Clinics in Chest Medicine 19:1 (1998).

A milder form of sleep disordered breathing affects many millions of people in the United States. Additionally, several million people suffer from an even more severe form of sleep disordered breathing (National Commission on Sleep Disorders Research, Bethesda, Md. (1995).

Pathophysiologically, snoring and sleep apnea are characterized by a recurrent closure of the pharyngeal airway during sleep. Upper airway patency is influenced by muscle activity, anatomical features, vasomotor tone, mucosal adhesive forces and inflammation (Clinics in Chest Medicine, 19:1 (1998)).

Snoring is an inspiratory sound which arises during a person's sleep. It is believed to be generally caused by the narrowing of the nasopharyngeal airway which is caused by a turbulent airflow during relaxed breathing which vibrates the soft parts of the oropharyngeal passage, such as the soft palate, the posterior faucial pillars of the tonsils and the uvula. While snoring is unpleasant for other people, it is typically not dangerous to the snorer and may cause fatigue. On the other hand, sleep apnea causes disruption in the sleep pattern and can result in daytime tiredness, loss of alertness and productivity. It would thus be advantageous to provide a treatment for both snoring and sleep apnea.

The current treatments of sleep apnea and snoring are dominated by both pharmacological and non-pharmacological treatments, however, none of these have been found entirely satisfactory.

Examples of nonpharmacological treatment include positive pressure therapy, such as nocturnal ventilation, continuous positive airway pressure, oral apparatuses, such as tongue retainers and jaw protractors, and surgical management, such as uvulopalatopharyngoplastic surgery comprising removal of accessory pharyngeal tissue. A comprehensive overview of these techniques is given in Clinics in Chest Medicine, 19(1):55-68 (1998); Clinics in Chest Medicine, 19(1):69-76 (1998); and Clinics in Chest Medicine, 19(1):77-86 (1998), among others.

Numerous other non-pharmaceutical treatment modalities have been proposed and used, however, these treatments, similar to those described above, are not entirely satisfactory and effective. Amongst these modalities are techniques used to manipulate a sleep position by, for example sewing a marble or tennis ball into a pyjama to avoid supine sleeping, visual or electric manipulation triggered by microphones or mild electrical shock devices, or mechanical devices used to manipulate the head position.

Other treatments utilize such conservative measures as weight loss, reduction of alcohol consumption and avoidance of medications which influence muscular tone.

Pharmacological treatment modalities include the systemic application of the therapeutic agents, such as tricyclic antidepressants, medroxyprogesterone acetate, tryptophane and other agents. All these agents have been used only with limited success, in part because they can cause undesirable secondary reactions.

Some attempts were made to treat and prevent snoring and sleep apnea with various topically administered agents. In this regard, to date, the following nasal spray applications have been suggested as possible treatments for snoring.

Phosphocholinamine as a topical spray (Am. J. Otolaryngol:, 8: 236 (1987)), topical administration of methylsulfonylmethane to the nasal epithelium (U.S. Pat. No. 5,569,679), and a mixture of surface active agents including Polysorbate 80, commercially available under the trade name Sonarex®, were suggested and/or are available as a topical spray for snoring.

The idea of nasal sprays to treat snoring dates back to 1955, when surface active substances, but not tyloxapol or alkylaryl polyether alcohol polymers, were first proposed for this purpose in U.S. Pat. Nos. 2,989,437 and 4,668,513 and in German patent 3,046,125. The patent application WO 98/46245 proposes use of phospholipid lung surfactants for treatment of sleep apnea.

Other proposed treatment for snoring include the use of mucopolysaccharides (U.S. Pat. No. 5,516,765), use of surfactant, preservatives and microbiocides (DE 3,917,109), pilocarpine (U.S. Pat. No. 5,502,067), a mixture of herbal enzymes (U.S. Pat. No. 5,618,543) and use of ubidecarone, a lipid existing in mitochondria (JP 1,165,522). U.S. Pat. No. 5,569,679 proposes using a solution of 1-20% methylsulfonylmethane along with an analgesic compound.

The inventors of U.S. Pat. No. 5,618,543 propose a mixture of natural enzymes and herbs as a remedy for snoring and allergies, given preferably as tablets. The U.S. Pat. No. 2,989,437 describes a combination of an anti-inflammatory and an anti-bacterial substance as a nasal decongestant which could decrease snoring. The U.S. Pat. No. 4,668,513 proposes, as a treatment for snoring, a composition comprising a surface active substance, a preservative, and a bactericidal or fungicidal substance in the form of a nasal spray.

None of the above treatments have been found to be effective for treatment of snoring and thus far none have been routinely utilized in practice.

Thus the need for effective, practical and non-invasive treatment of snoring persist.

Alkylaryl polyether alcohol polymers and particularly tyloxapol are compounds which are known for their mucolytic activity and have been previously used for inhalation treatment of lung inflammation. These compounds are generally classified as dispersants.

The U.S. Pat. No. 5,849,263 describes a pharmaceutical composition containing from 0.125% to 5% of tyloxapol useful for inhalation purposes, and suggests strategies to reduce hypertonicity to avoid bronchospasm upon inhalation into the lung. Other related proposals for the use of tyloxapol are as a treatment for lung inflammation associated with cystic fibrosis (Australian Patent AU 717 537), pulmonary inflammation (WO 97/38 699), and as an anti-oxidant (U.S. Pat. No. 5,512,270).

Specifically, the above described prior inventions relate to aerosol treatments of respiratory inflammation and cystic fibrosis. The inventors describe in a detailed fashion the oxidant-mediated injury in the lung, the effect of hydroxyl group(s), other free radicals, cytokines and inflammatory parameters. These factors, in combination with hyperviscous mucous production, play a role in cystic fibrosis.

While some of these patents disclose the use of tyloxapol aerosol in the pulmonary diseases, and briefly mention its possible use for relief of nasal rhinitis, rhinosinusitis or other inflammation, they do not describe, disclose or suggest a possible use of tyloxapol for treatment of snoring, sleep apnea or improvement of nasal breathing.

The compounds which are subject of this invention have never before been used or their use suggested for treatment of snoring and/or sleep apnea and/or sudden infant death syndrome and/or improvement of nasal breathing.

WO98/46245 proposes administration of phospholipid lung surfactants containing minute amounts of dispersant tyloxapol to the posterior pharyngeal region prior to sleep in order to reduce sleep apnea. The described active compounds are natural or synthetic lung surfactants rather than dispersants and antioxidants. The application does not teach the use of a nasal spray and the use of tyloxapol for treatment of snoring.

The current invention is based on a discovery that tyloxapol and related alkylaryl polyether alcohol polymers can decrease, prevent or treat snoring, sleep apnea, sudden infant death syndrome and sleep disturbances connected therewith in humans as well as improve nasal breathing following physical exertion, impaired breathing or post-surgical breathing trauma in mammals.

None of the above described disclosures teaches the current invention of administering tyloxapol or related alkylaryl nasally and/or pharyngeally to treat snoring and sleep apnea, to prevent sudden infant death syndrome and to improve nasal breathing.

Use of tyloxapol or related alkylaryl polyether alcohol polymers has never been proposed as a treatment for snoring and/or sleep apnea, or as a method to improve nasal breathing. Alkylaryl polyether alcohol polymers such as tyloxapol are known to be active as mucolytics, antioxidants, free radical scavengers, and as dispersant agents. This group of compounds is distinct from the other compounds previously used or proposed for use in treatment of snoring and sleep apnea and other diseases and conditions described herein.

The current invention specifically describes the use of topical nasal and pharyngeal compositions comprising one or several alkylaryls for treatment of snoring, sleep apnea, sudden infant death syndrome and improvement of nasal breathing.

All patents, patent applications and publications described herein are hereby incorporated by reference.

### SUMMARY

One aspect of the current invention is a method for treatment or prevention of snoring, sleep apnea, sudden infant death syndrome and sleep disorders and for improvement of sleep pattern, alertness and nasal breathing by administering to a subject in need thereof a composition comprising from about 0.01 to about 20% of alkylaryl polyether alcohol polymer or a combination thereof with or without admixture with a pharmaceutically acceptable excipient or additive.

Another aspect of the current invention is a method for prevention and treatment of snoring in humans by administering to a subject in need thereof a composition comprising from about 0.2 to about 20% of alkylaryl polyether alcohol polymer or a combination thereof.

Still another aspect of the current invention is a method for prevention and treatment of snoring in humans by administering to a subject in need thereof a composition comprising from about 1 to about 10% of tyloxapol alone or in admixture with a pharmaceutically acceptable excipient and/or additive administered nasally and/or pharyngeally prior to or during sleep.

Still another aspect of the current invention is a method for prevention and treatment of snoring in humans by administering to a subject in need thereof from about 0.045 to about 3 ml of a nasal/pharyngeal spray comprising from about 1 to about 100 mg/ml of tyloxapol alone or in admixture with a pharmaceutically acceptable excipient and/or additive administered nasally and pharyngeally prior to sleep up to a total daily dose of 3 grams.

Yet another aspect of the current invention is a method for treatment and prevention of sleep apnea in humans by administering to a subject in need thereof a composition comprising from about 0.5 to about 20% of one or a combination of several alkylaryl polyether alcohol polymers.

Still another aspect of the current invention is a method for prevention and treatment of sleep apnea in humans by administering to a subject in need thereof a composition comprising from about 0.5 to about 20% of tyloxapol alone or in admixture with a pharmaceutically acceptable excipient and/or additive administered nasally and/or pharyngeally prior to or during sleep.

Still yet another aspect of the current invention is a method for prevention and treatment of sleep apnea in humans by administering to a subject in need thereof from about 0.045 to about 3 ml of a composition comprising from about 10 to about 150 mg/ml of tyloxapol alone or in admixture with a pharmaceutically acceptable excipient and/or additive administered nasally and/or pharyngeally prior to or during sleep up to a total daily dose of 3 grams.

Another aspect of the current invention is a method for prevention of sudden infant death in infants comprising administering a composition comprising alkylaryl in concentration from about 0.01 to about 5% of selected alkylaryl administered to a nostril of an infant before sleep one or several times a day.

Still yet another aspect of the current invention is a method for prevention and treatment of sudden infant death syndrome in infants by administering to an infant from about 0.015 (1 drop) to about 0.5 ml of a composition comprising from about 0.1 to about 50 mg/ml of tyloxapol alone or in admixture with a pharmaceutically acceptable excipient and/or additive administered to an infant nasally prior to or during sleep one or several times a day up to a daily dose of 1 gram.

Still yet another aspect of the current invention is a method for improvement of sleep pattern, treatment of sleep disorders and for improvement of day alertness by administering to a subject in need thereof a nasal spray or another composition comprising from about 0.2 to about 20% of tyloxapol alone or in combination with pharmaceutically acceptable excipients and/or additives.

Still yet another aspect of the current invention is a method for improvement of nasal breathing during and following the physical performance such as competitive sports, diving, flying, high altitude climbing, horse racing, etc., in mammals, including humans, or improving nasal breathing in mammals having anatomically or functionally impaired nasal passageways by administering to a subject in need thereof a nasal spray composition comprising from about 0.2 to about 20% (2-200 mg/ml) of tyloxapol alone or in combination with pharmaceutically acceptable excipients and/or additives prior to or following the physical performance up to a daily dose of 3 grams for humans and more than 10 grams for large animals.

Still yet another aspect of the current invention is a composition comprising one or a combination of several alkylaryl polyether alcohol polymers having a structure of general formula

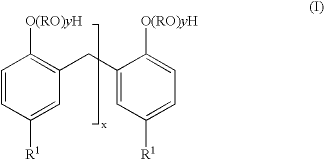

wherein R is ethylene, R¹ is tertiary octyl, X is greater than 1, and Y is an integer from 8 to 18, or a pharmaceutically acceptable salt thereof.

Yet another aspect of the current invention is a composition comprising tyloxapol having a general formula

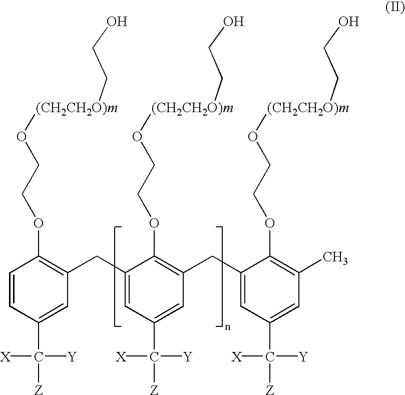

wherein X is hydrogen or methyl, Y is hydrogen or methyl, Z is hydrogen or straight or branched hydrocarbon chain of 1-8 carbons, m is an integer from 6-8 and n is an integer equal to or smaller than 5, or a pharmaceutically acceptable salt thereof.

Still another aspect of the current invention is a spray, liquid or solid nasal or pharyngeal composition comprising from about 0.01 to about 20%, that is from about 0.1 to about 200 mg/ml, of tyloxapol or another alkylaryl polyether alcohol polymer per one ml of a diluent for nasal administration as a nasal and/or pharyngeal spray, nasal and/or pharyngeal solution, nasal and/or pharyngeal drops, lozenges, nasal aerosol or dry powder, administered directly, or by using a device for nasal or pharyngeal administration.

Another aspect of the current invention is a metering dose device for administration of the composition of the invention in predetermined dose.

Definitions

As used herein:

“Alkylaryl” means alkylaryl polyether alcohol polymer depicted by formula (I).

“Tyloxapol” means a compound depicted by formula (II).

“Active component”, “active compound” or “active ingredient” means one of the alkylaryl polyether alcohol polymers, preferably tyloxapol, as defined above.

“CPAP” or “continuous positive airway pressure” means continuous positive airway pressure treatment for snoring and sleep apnea which is typically administered via the nose (nCPAP) or the mouth of the patient.

“TNS” means tyloxapol nasal spray.

“SID” or “SIDS” means sudden infant death syndrome.

“OSAS” means obstructive sleep apnea syndrome.

“Normal saline” or “NS” means water solution containing 0.9% (w/v) NaCl.

“Diluted saline” means normal saline containing 0.9% (w/v) NaCl diluted into its lesser strength from about 0.1% to about 0.45%.

“Half normal saline” or “½ NS” means normal saline diluted to its half strength containing 0.45% (w/v) NaCl.

“Quarter normal saline” or “¼ NS” means normal saline diluted to its quarter strength containing 0.225% (w/v) NaCl.

“One tenth normal saline” or “1/10 NS” means normal saline diluted to its one tenth strength containing 0.09% (w/v) NaCl.

“AHI” means apnea/hypopnea index.

“VAS” means visual analog scale.

“RDI” means respiratory distress index.

“Squirt” means a volume dose of approximately 0.14 ml.

“Drop” means a volume dose of approximately 0.015 ml.

### BRIEF DESCRIPTION OF FIGURES

FIG. 1 is a graph illustrating decrease in snoring loudness following treatment with tyloxapol as determined by the visual analog scale (VAS).

FIG. 2 is a graph showing decrease in occurrence of apneic/hypopneic episodes in sleep apnea patients following treatment with tyloxapol as determined by apnea hypopnea index (AHI).

FIG. 3 is a graph illustrating improvement in sleep following treatment with nasal spray containing 1% of tyloxapol in sleep apnea patients, determined as sleep efficiency (SE).

FIG. 4 is a graph illustrating improvement of sleep in sleep apnea patients following treatment with 1% tyloxapol nasal spray, measured by number of arousals per hour (ArI).

### DETAILED DESCRIPTION OF THE INVENTION

The current invention concerns methods and compositions for treatment and prevention of snoring and sleep apnea in humans, for prophylaxis of sudden infant death syndrome in infants, or for general improvement of sleep pattern and nasal breathing, for treatment, pretreatment and improvement of performance in a human or animal subjects prior to, during or following the physical performance.

The methods for treatment of the above conditions are efficient, safe, non-invasive and convenient. The treatment is achieved by providing a subject with an easy to administer composition of the invention, said composition comprising one or a combination of several alkylaryl polyether alcohol polymers formulated as a spray, liquid or solid composition for nasal and/or pharyngeal administration.

Upon nasal and/or pharyngeal application of the composition prior to or during sleep according to appropriate regimens, the incidence and severity of snoring and sleep apnea is reduced, sudden infant death in infants is prevented and nasal breathing is improved in mammals with anatomically or functionally obstructed nasal passageway or before, during or following a physical activity or competitive sports, such as diving, high altitude climbing, hiking or flying in humans, or horse or dog racing, etc. in animals. Additionally, the method according to the current invention substantially improves daytime alertness and performance.

I. Compounds of the Invention

Compounds of the invention are known for their activity as dispersants, mucolytics, antioxidants, anti-inflammatories and free radical scavengers.

A. Chemical Characterization

Active compounds of the invention are alkylaryl polyethers alcohol polymers represented by the general chemical formula

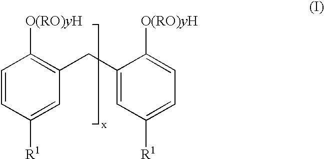

wherein R is ethylene, R¹ is tertiary octyl, X is greater than 1, and Y is an integer from 8 to 18, or a pharmaceutically acceptable salt thereof.

Alkylaryl polyether alcohol polymers are a well known group of mucolytic dispersants. Representative compounds are tyloxapol, Triton WR-1352, Triton M-3610, Triton N-100, Triton N-155, Triton WR-1360, Triton WR-1363, Triton WR-1369, WR-1364. Processes for preparation of these compounds are known in the art.

B. Pharmacological Characterization

Alkylaryl polyether alcohol polymers of the invention have a pharmacological activity as dispersants, mucolytics, antioxidants, anti-inflammatories and free radical scavengers when topically applied to the epithelium of the upper airways.

The mode of action of alkylaryl polyether alcohol polymers resulting in a decrease or cessation of snoring and sleep apnea can be described in both physical and pharmacological terms.

Physically, the alkylaryls dispersant action was found to reduce the collapse of muscular and epithelial structures in the nose and throat, thereby improving upper airway patency during inspiration.

The pharmacological activity of alkylaryls was found to result in reduction of inflammation and in protection of the nasal and pharyngeal epithelium from swelling and damage. Since alkylaryls are not well absorbed systemically, pharmacological activity of alkylaryls affecting snoring and sleep apnea is due to a direct topical effect on the collapsing epithelium of the upper airways.

C. Tyloxapol—Chemical Characterization

The most preferred alkylaryl polyether alcohol polymer is tyloxapol, represented by the chemical formula (II)

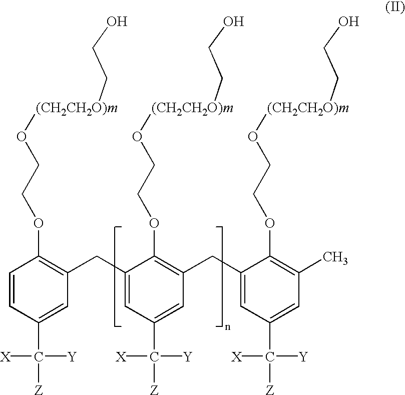

wherein X is hydrogen or methyl, Y is hydrogen or methyl, Z is hydrogen or straight or branched hydrocarbon chain of 1-8 carbons m is an integer from 6-8 and n is equal or smaller than 5, or a pharmaceutically acceptable salt thereof.

Tyloxapol is a known compound previously disclosed in U.S. Pat. No. 2,454,541 as a mucolytic dispersant. Tyloxapol, also known and available under the trade names Triton WR-1339, Triton A-20, Superinone, Alevaire®, or Tacholiquin® is listed in Merck Index under a chemical name as an oxyethylated tertiary octylphenol formaldehyde polymer, an oxyethylated tertiary octyl-phenol-polymethylene polymer or a p-isooctylpolyoxyethylenephenol formaldehyde polymer. Tyloxapol is a blend of alkylaryl polyether alcohol polymers fitting within the formula II. Tyloxapol USP can be purchased from Ruger Chemical Company, Inc., Irvington, N.J. 07111 and is also commercially available from Organichem, Rensselaer, N.Y.

Tyloxapol is a viscous compound, miscible with water at all concentrations and soluble in the majority of organic solvents. Tyloxapol is a chemically stable compound unaffected by boiling, sterilization, extensive length storage or prolonged standing and is compatible with various buffers, buffer salts and a wide variety of organic compounds without changing its chemical characteristics.

Tyloxapol has a dispersant and mucolytic activity on mucosal tissue.

Tyloxapol has been used in humans as a treatment for a variety of pulmonary disorders, primarily for treatment of tuberculosis and as an aerosolized agent for treatment of bronchitis, asthma, respiratory distress and bronchiectasis, or as a dispersant for other pharmacologically active substances. Tyloxapol has been shown to be poorly absorbed from the gastrointestinal tract and its intravenous administration results in hyperlipemia.

Tyloxapol has never before been used for nasal or pharyngeal administration to treat snoring or sleep apnea or other conditions as described herein.

D. Tyloxapol—Pharmacological Characterization

Tyloxapol, as an example of alkylaryl polyether alcohol polymers, is known as a mucolytic compound reducing epithelial secretions, viscosity and tenacity of the sputum.

It has been used for a number of years as an aerosolized tyloxapol, available under a product name Alevaire®, administered in an inhalable nebulized form for treatment of bronchitis and tracheitis. The current pharmaceutical utility for tyloxapol, which is now marketed and approved for use only in Japan and Germany, is only for aerosol administration to the lung by a nebulizer.

The use of tyloxapol as a nasal spray for treatment of snoring and sleep apnea, prevention of sudden infant death syndrome or improvement of nasal breathing and sleep pattern has never before been disclosed.

It has now been discovered that a composition comprising tyloxapol is suitable for treatment and prevention of snoring, sleep apnea, sudden infant death syndrome or for general improvement of nasal breathing during physical activity or medical conditions when used as a nasal and/or pharyngeal spray, liquid, lozenge, dry powder or nasal aerosol.

II. Compositions of the Invention

Composition of the invention consist essentially of an active ingredient and covers all pharmaceutically acceptable formulations containing alkylaryl polyether alcohol polymers, preferably tyloxapol, as an active ingredient for the treatment of snoring and/or sleep apnea and/or other conditions described herein.

The pharmaceutically acceptable formulations comprise a selected alkylaryl or tyloxapol at concentrations ranging from 0.01% to 20% (0.1 to 200 mg/ml) with the preferable range for each condition being from about 0.2% to about 10% (2 to 100 mg/ml) for treatment of snoring, from about 0.5% to about 15% (5 to 150 mg/ml) for treatment of sleep apnea, from about 0.01% to about 5% (0.1 to 50 mg/ml) for prevention of sudden infant death syndrome, and from about 0.2% to about 20 (2 to 200 mg/ml) for improvement of alertness and physical performance.

The composition of the invention is typically administered as a nasal or pharyngeal spray although it may be administered as a liquid, liquid drops, lozenge, tablet, nasal aerosol or dry powder.

The composition comprises one or a combination of two or more compounds selected from the group of alkylaryl polyether alcohol polymer compounds depicted by formula (I). The most preferred alkylaryl is tyloxapol depicted by the formula (II).

The selected alkylaryl is present in from about 0.01 to about 20%, that is from about 0.1 to about 200 mg/ml, depending on the intended use.

The composition intended for treatment of snoring comprises from about 0.2 to about 20%, preferably from about 1 to about 10%, and for treatment of sleep apnea from about 0.5 to about 20%, preferably from about 1 to about 15%, of alkylaryl, preferably tyloxapol up to a maximum of 3 grams per day.

The composition intended for treatment and prevention of sudden infant death syndrome comprises from about 0.01 to about 5% of alkylaryl, preferably tyloxapol up to a maximum of 1 gram per day.

The alkylaryl of the invention is formulated as a spray, liquid, drops, lozenge, nasal aerosol or dry powder alone or in admixture with any suitable pharmaceutically acceptable excipient and, when appropriate, is diluted in a pharmaceutically suitable diluent, such as a sterile water, normal or half or quarter diluted saline or another, preferably aqueous, diluent.

The alkylaryl polyether alcohol polymers may also be used in combination with other topically active agents alone, as a combination of the alkylaryl and the topically active agent (s), such as antibiotics, anti-inflammatories, analgesics and some such other compounds, or in admixture with any suitable pharmaceutically acceptable excipient.

The composition is prepared and administered in a dose form per treatment formulation comprising from about 0.01 to about 200 mg of active component, typically applied to humans in a volume of 0.045 ml corresponding to about 2-3 drops to about 3 ml, preferably about 0.2 to about 1 ml per nostril or pharyngeally per one treatment. Typically, the volume administered into the nostrils is smaller than the volume administered pharyngeally. The administered volume depends on the patient and condition treated such that for infants, for example, the volume is selected to barely coat the pharyngeal region to prevent the aspiration of the solution to the lungs or swallowing the excess of the solution. In adult patients, the administered volume may be larger as the patient is not likely to aspire the excess of the solution, and the area to be coated is also much larger. Volume used for treatment of animals depends on the size of the animal nasopharyngeal area.

The dose per treatment depends on the intended use and typically comprises from about 0.1 to about 200 mg. The dose for treatment of snoring is typically from about 2 mg/ml to about 200 mg/ml, preferably from about 10 mg/ml to about 100 mg/ml of active component whereas the dose for treatment of sleep apnea is from about 5 mg/ml to about 200 mg/ml, preferably from about 10 to about 150 mg/ml, for improvement of sleep pattern, increase of alertness and improvement of breathing the dose per treatment is from about 2 to about 200 mg, preferably about 5 to about 150 mg/ml and the dose for prevention of sudden infant death is from about 0.1 to about 50 mg/ml, preferably from about 0.5 to about 10 mg/ml.

The composition of the invention is prepared, supplied and stored preferably under sterile conditions. The composition may be sterilized by any available, acceptable or suitable sterilization technique which does not affect and/or destroy the activity of the active compound. Preferred sterilization technique is filtration. Sterilization of the tyloxapol solution, for example is advantageously accomplished by vacuum filtration through a filter, such as Millipore filter. Following the sterilization process, the sterile composition is packaged into sterile nasal spray bottles, glass vials or another suitable container for liquid or solid formulation.

The storing of the composition depends on the formulation. For solutions, spray or drops, the composition is stored in a glass or plastic, such as a polypropylene or polyethylene container, which may be clear or colored, containing applicators, nozzles, droppers, pipette, metered pump with either a throat or nasal actuator or propeller, as appropriate. The container is closed and protected from outside contamination by a closure system, such as a top, crimp or snap-on.

A. Specific Formulations

For each intended use, the alkylaryl, preferably tyloxapol, is formulated to meet specific criteria of the treated conditions or route of delivery.

1. Spray Formulation

Nasal or pharyngeal spray formulation comprises from 0.01 to 20% of a selected alkylaryl dissolved in sterile water, normal saline, diluted saline, preferably in quarter diluted saline. Glycerol, sodium bicarbonate, sodium chloride, potassium chloride or calcium chloride etc., may be optionally added in appropriate percentage concentration.

One specific formulation, for example, comprises 10 mg of tyloxapol, 50 mg glycerol and 20 mg of sodium hydrogen per 1 ml of sterile water.

Typical drug dosage of the spray formulation is two-three squirts per nostril and three squirts to the upper pharynx. One squirt comprises about 0.14-0.15 ml/spray. A patient thus receives six squirts nasally and three squirts pharyngeally corresponding to a total dosage of approximately 1.26-1.35 ml of the spray. If, for example, the spray contains 10 mg of tyloxapol per 1 ml, the patient receives 12.6 to 13.5 mg of tyloxapol as one total dose.

Tyloxapol spray formulations is prepared by dissolving tyloxapol or another active component, or a combination of two or more in an appropriate diluent under sterile conditions and introduced into a spray container equipped with an appropriate actuator, preferably metered dose actuator which measures the dose administered as one squirt.

Devices suitable for delivery of spray formulation are described below.

2. Liquid Formulation

Liquid nasal or pharyngeal formulation is essentially the same as the spray formulation. The dosage of the active component is adjusted for administration as drops. One drop of the formulation corresponds to about 15-20 microliters (15 μl). Typical one dosage is about 1 to 30 drops corresponding to from about 15-200 μl (0.015-0.2 ml) to about 450-600 μl (0.45-0.6 ml) administered to each nostril.

Although the liquid or liquid drops may be administered also pharyngeally, it is preferred that the liquid is applied to nostrils from where it seeps to pharynx.

In the alternative, the nasal drops or nasal solution may also be applied via a nasal catheter inserted in the nose. The solution is applied through this catheter reaching the back of the nasal passages from the nasal floor.

The liquid formulation thus comprises such a dosage of the active component which is measurable in drops amounts.

The formulation is stored in glass or plastic vials or containers which are equipped with a dropper.

3. Lozenges, Tablets, Troches

Alkylaryl, preferably tyloxapol, may also be conveniently formulated as lozenges, tablets or troches. The tyloxapol containing suckable/masticable lozenge is an oral lozenge from which the active compound is released to the velopharynx over time. The pharmaceutical lozenge formulations according to the present invention are used in the treatment of snoring, sleep apnea, and upper airway patency.

Typically, a lozenge composition containing a therapeutically effective amount of alkylaryl polyether alcohol polymer, preferably tyloxapol, releases the active component into the oral cavity so as to deliver the active component to the surface of the velopharynx and upper airways. The lozenge is used before sleeping, preferably within 30-60 minutes before falling asleep.

The drug dosage form is provided as a lozenge or suckable tablet which is intended to be sucked by the patient. The term lozenge as used herein is intended to embrace all dosage forms where the product is formed by cooling or consolidating a sugar or sugar alcohol based molten mass containing the tyloxapol or another alkylaryl. A volatile substance, such as menthol or eucalyptus oil, may be added to facilitate deposition of the active drug to the upper airways and velopharynx. The term tablet as used herein is intended to embrace unit dosage forms made from compressed powders or granules or compressed pastes.

Solid dosage forms, i.e., lozenge and tablets, are prepared by methods well known in the art for the production of lozenges, tablets, capsules or chewing gums and may contain other ingredients such as acidity regulators, opacifiers, stabilizing agents, buffering agents, flavorings, sweeteners, coloring agents, astringent, antiseptics, and preservatives.

A typical lozenge, tablet or troche is composed predominantly of an inert vehicle, carrier, or diluent. The medicinal agent is interspersed within this carrier. When placed in the oral cavity, the lozenge, tablet or troche will slowly dissolve thereby releasing the active component so that it comes in contact with the tissues of the mouth and upper throat.

Lozenge formulations are known and are typically used to treat conditions such as throat infections, dental plaque, halitosis, etc. The current invention utilizes modified lozenges containing one or a combination of several alkylaryls.

Further detail of lozenge formulations can be found in patents U.S. Pat. No. 5,322,694, U.S. Pat. No. 6,194,003, U.S. Pat. No. 5,700,514 and U.S. Pat. No. 6,166,083, incorporated herein by reference.

4. Powder Formulation

Alkylaryl polyether alcohol polymer, preferably tyloxapol, may be advantageously applied to the velopharyngeal area and upper airways as a dry powder, without significant lung deposition.

This type of treatment requires that a powder has particle sizes larger than 5 microns, preferably between 5 and 100 microns. These particles are easy to deposit to the velopharynx and upper airways but are not delivered and deposited to the lower airways and lung.

The alkylaryl polyether alcohol polymer compounds of the invention may be administered to the upper airways and velopharynx in a dry powder formulation or by metered dose inhalers as an alternative therapy to topical nasal/throat spray or aerosol delivery.

Dry powder inhalation and metered dose inhalation are more practical when administered doses result in the delivery of at least about 2-5 mg, and more preferably about 10 to about 200 mg, of alkylaryl polyether alcohol polymer compound to the upper airways of the patient receiving treatment.

In this aspect, the invention provides a sufficiently potent formulation of alkylaryl polyether alcohol polymer, preferably tyloxapol, in dry powder or metered dose form of drug particles milled to particle sizes predominantly with a range of 5 to 100 microns.

For dry powder formulations of the invention, an alkylaryl polyether alcohol polymer, preferably tyloxapol, is milled, spray dried, lyophilized or otherwise processed to a powder having mass median average diameters ranging from 5 to 100 microns by media milling, jet milling, spray drying, particle precipitation techniques or lyophilization. Particle size determinations may be made using a multi-stage Anderson cascade impactor or other suitable method.

Media milling may be accomplished by placing the alkylaryl into a mill containing, for example, stainless steel or ceramic balls and rotating or tumbling the material until the desired drug particle size ranges are achieved.

Advantages of media milling include good size control, narrow product size ranges, high efficiencies of recovery, and readily scalable processes. Disadvantages include long process times (on the order of hours to days), the requirement that the milling media be separated from the product at completion, and the possibility of contamination of the product with the media.

Alternatively, the dry powder formulations may be prepared by jet milling techniques. Jet milling uses very high pressure air streams to collide particles with one another, with fine particles of the desired size being recovered from the mill. Advantages include rapidity (seconds to minutes for completion) and less energy transfer during milling resulting in less temperature rise of drug product. Disadvantages include poorer collections efficiencies of 50 to 80% recovery. Both techniques and any and all improvements thereof are intended to be within the scope of the invention.

In other embodiments, the dry powder formulations may be prepared by spray drying, solution precipitation or lyophilization techniques. Spray drying is achieved by spraying a fine mist of drug solution onto a support and drying the particles. The particles are then collected. Spray drying has the advantage of being the least prone to degrading chemical entities. Adding a co-solvent that decreases the solubility of a drug to a uniform drug solution performs solution precipitation. When sufficient co-solvent is added, the solubility of the drug falls to the point where solid drug particles are formed which can be collected and separated by size by filtration or centrifugation. Precipitation has the advantage of being highly reproducible and can be performed under low temperature conditions, which reduces degradation. Dry powder prepared by lyophilization utilizes isolation of solid dry powder from solution comprising alkylaryl by freezing the solution and evaporating the ice under vacuum.

The dry powder formulations of the invention may be used directly in metered dose or dry powder inhalers. There are two major designs of dry powder inhalers. Device-metering designs contain a reservoir in which active compound is stored within the device and the patient “loads” a dose of the compound into the inhalation chamber. Factory-metered devices contain a separate container in which each individual dose has been already manufactured.

Drug powder is placed into the inhalation chamber (either by device metering or by breakage of a factory-metering dosage) and the inspiratory flow of the patient accelerates the powder out of the device and into the oral cavity or, if the device is equipped with a nasal catheter, inspired into the nostril.

Non-laminar flow characteristics of the powder path cause the excipient-drug aggregate to decompose, and the mass of the large particles causes their impaction inside the nostril or at the back of the throat. Current technology for dry powder inhalers is such that payload limits are around 50 mg of dry powder of which the drug is usually only a partial component by mass.

Effective dosage levels of alkylaryl polyether alcohol polymers for dry powder inhalation and metered dose inhalation result in the delivery of at least about 2-5 mg, and more preferable about 10 to about 200 mg of alkylaryl polyether alcohol polymers to the upper airways of the patient receiving treatment.

Depending on the efficiency of the dry powder delivery device, dry powder formulations suitable for use in the invention comprise from about 2 to about 200 mg, preferably from about 5 to about 100 mg of powder in particle sizes above 5 microns in mass median average diameter necessary for deposition at the velopharynx and upper airways. The dry powder formulation may be delivered prior to sleep, or several times during the night or before physical activity.

The dry powder formulations have a physiologically acceptable pH of 4.0 to 7.5, preferably 6.5 to 7.0, are temperature stable and have the advantage of a long shelf life.

B. Devices

The composition of the invention is preferably administered in a spray or liquid form using containers, vials, or small portable devices such as pumps, atomizers, propellant tubes, masks or dry powder or metered dose inhalers.

1. Spray Containers, Vials and Pumps

A spray or liquid formulation is administered from spray containers, vials or spray pumps.

One example of the spray device is a container for instillation of a metered quantity of solution into the nasal passageway. Such container may be a bottle, vial, tube, etc., for holding the solution, typically 10-100 ml volume, which includes an applicator comprising a metering structure, typically activated by a plunger. Upon depressing the plunger, a metered quantity of the solution is ejected through a nozzle placed in the nostril.

The device may be sterilized before the sterile solution is added and is impregnably enclosed to keep the solution sterile. However, non-sterile version may also be used.

Another type of device used preferably for administration of liquid formulation is a plastic squeeze bottle, adapted to hold fluid to be dispensed. The plastic squeeze bottle is in communication with a nozzle used for nasal instillation of the liquid formulation. This type of device is calibrated to deliver dosages in drops or squirts. The bottle may be held upright with the nozzle positioned in the nostril to deliver atomized mist ejected from the nozzle upon squeezing the bottle.

Alternatively, squeeze bottle may be used to deliver drops of the formulation by squeezing the inverted bottle into the nostril.

Another type of device which is suitable for administering the liquid formulation is a standard eye dropper mounted into a cap for closing a bottle containing the formulation.

In this regard, devices suitable for use in this invention are modified devices described, for example, in U.S. Pat. No. 5,569,679.

As an alternative to the nasal spray application, the alkylaryl polyether alcohol polymer, preferably tyloxapol, may also be applied to the velopharynx and upper airways via the mouth, i.e. by a throat spray.

Device used for such purpose is, for example, a pump spray device with an extension nozzle 3 to 9 cm in length, able to reach the posterior part of the mouth), preferably with a spray angle that is directed in a 30 to 60 degree angle upwards. Such device is used to efficiently apply the active compound to the collapsible, upper airways. The throat spray improves the topical deposition of the composition directly to the velopharynx by circumventing the filtering abilities of the nasal passages.

Analogously, a simple nasal instillation by a nasal/oral catheter may be used instead of the nasal/throat spray.

2. Atomizers, Nebulizers, Aerosolizers, Humidifiers

Another type of device which is advantageously utilized for delivery of alkylaryls into the velopharynx and upper airways are atomizers, nebulizers, aerosolizers or humidifiers. Using these devices, the active component of the invention is atomized, nebulized or aerosolized using devices known in the art used for delivery of drugs into the lower airways and to the lung.

For purposes of the current invention, the active component is atomized, nebulized or aerosolized into particle sizes between 5 and 100 microns. To achieve this, the atomizers, nebulizers or aerosolizers are equipped with separation buffels which separate, remove and recycle particles smaller than 5 microns because these would be deposited in the lower airways and lungs, which for the purpose of this invention, is undesirable.

Any suitable device and apparatus which can generate particles above 5 and below 100 microns of a selected alkylaryl is suitable for nasal, pharyngeal and velopharyngeal delivery of the composition of the invention.

To name a few representative apparatuses, the aerosolizers described in U.S. Pat. No. 5,849,263, commercial nebulizers, aerosol or spray mechanical pump, coarse liquid spray, colloidal suspension spray, liquid droplet or liquid droplet suspension in the carrier producing apparatuses and devices, gaseous carrier devices or humidifiers, as described, for example, in U.S. Pat. Nos. 5,653,919, 6,325,063, 6,293,279 and 6,237,591 are all useful in carrying out this invention as long as the produced particles are predominantly, that is at least 90% of particles, are within the confine of from about 5 to about 100 microns.

3. Masks

The pharmaceutical formulations provided by the present invention are intended to be used in the treatment of snoring, sleep apnea, and improvement of upper airway patency by the administration to a patient in need of such treatment a composition comprising tyloxapol or another alkylaryl. Besides spray, aerosol or lozenge administration, the mode of administration also includes applications via nasal and oral masks, especially through those commonly used in continuous positive airway pressure (CPAP) or nose (nCPAP) treatment.

The CPAP treatment for snoring and sleep apnea according to the invention typically comprises administration of the composition of the invention via the nose (nCPAP) or the mouth of the patient.

The treatment of the current invention can be given prior to or along with the CPAP treatment. The parallel application of an effective amount of alkylaryl polyether alcohol polymer, preferably tyloxapol, can be accomplished by adding an additional aerosol channel to the air pressure treatment.

Alternatively, the effective amount of alkylaryl polyether alcohol polymer, preferably tyloxapol, can be added to the water or into the liquid reservoir that is typically part of the CPAP treatment device.

As a synergistic effect, the alkylaryl polyether alcohol polymer, preferably tyloxapol, may reduce the required treatment pressure that is needed to prevent collapse of the upper airways.

Using mask treatment, the active component can be administered in a low concentration throughout the night, or, at higher concentrations, as a bolus, at different time points in the beginning and during the course of the night.

III. Pharmaceutically Acceptable Excipients

The composition of the invention may comprise any pharmaceutically acceptable excipient and/or additive suitable for nasal or pharyngeal administration.

Nasal or pharyngeal solutions are prepared in such a way that they resemble nasal secretions so that normal ciliary action is maintained. The composition is formulated to approximate the body's natural salinity and electrolyte conditions and for maximum upper airway tolerance. This includes, but is not limited to, the addition of sodium chloride, potassium chloride and calcium chloride for isotonicity, sodium phosphate for buffering the composition to the close to the physiological pH, to adjustment of osmolality, particle stabilization, antimicrobial agents, drug stabilizers etc.

The pH of the compositions is adjusted to be in range from 5.0 to 8.0 with the optimal and preferred pH from about 5.5 to about 6.5. The pH may be adjusted with, but not limited to, sodium bicarbonate, mono and dibasic phosphate, sodium hydroxide and hydrochloric acid. Any other suitable acid, base, buffer or buffering compound may be used for adjusting the pH of the composition to the above range.

It is preferred that the composition of the invention is isotonic or as close to isotonic as possible. While both sodium bicarbonate and glycerol may be added for certain types of compositions, they are known to increase the osmolality, thus making the solution hypertonic.

Glycerol, which is a stabilizing agent for certain particle containing formulations, may be optionally added or may be omitted from the formulation.

Glycerol, when added to the formulation, is added at a concentration ranging from about 0.1% to about 5%. The function of the glycerol is to stabilize particle formulation and distribution when the solution is aerosolized or delivered as a spray or liquid.

The optimization parameters of the formulation for airway tolerance include adjustment of osmolality to between about 150 to 550 mOsm/kg and the presence of a freely permeant anion, such as for example, chloride anion present from about 31 to about 300 mM.

Consequently, the active ingredient, such as tyloxapol or another alkylaryl, is preferably dissolved in a solution of between quarter normal saline, i.e. containing 0.225% NaCl and a normal saline containing 0.9% NaCl. Sodium chloride may be substituted with potassium chloride, calcium chloride or other pharmaceutically acceptable salts which are non-irritating to the mucosa and epithelium. In the alternative, tyloxapol or another alkylaryl may be dissolved in oil-in-water solution with the understanding that the oil portion is limited to minimal amounts intended solely for enhancing tyloxapol formulations. One example of a solution suitable for dilution of the active ingredient is Locke-Ringer's solution which is known to be well tolerated by the nasal epithelium.

Additives are compounds such as preservatives, colorants, stabilizers, antimicrobial agents etc. Preservatives, in this instance, may or may not be needed in the solution, depending on the active ingredient. For example, tyloxapol (commercially available and containing sodium bicarbonate and glycerol) itself is known as a preservative having the shelf life at room temperature of more than 6 years.

If found to be needed, a preservative suitable for nasal sprays are selected from benzalkonium chloride, parabens, thimerosal, disodium edetate, monobasic and dibasic sodium phosphate, potassium phosphate, phenylcarbinol, povidone and sodium silocoaluminate.

IV. Mode of Administration

The mode of administration and/or application of the composition of the invention comprising alkylaryl polyether alcohol polymer in a spray form to the velopalate, that is directly and specifically to the nasal and pharyngeal mucosa, is crucial for its efficacy.

Consequently, the composition is administered in two different modes, namely to the nasal mucosa (antegrade) and to the pharyngeal mucosa (retrograde). The composition can be administered solely to the nasal mucosa, or solely to the pharyngeal mucosa but is preferably administered to both as a part of one therapeutical dosage.

As described above, compositions of the invention are administered as a spray, as a liquid, as liquid drops, in a solid form as a lozenge, troche or tablet, or as aerosolized, nebulized or atomized solution. In all these modes, the composition is delivered to nasal or pharyngeal mucosa and preferably to both. Any combination of treatments and modes of administration is contemplated and intended to be within the scope of this invention. Thus, the lozenge delivery to pharyngeal area may be advantageously combined with nasal drops or spray, or nasal drops may be combined with pharyngeal spray, etc.

Regarding the spray application, both the direct, antegrade application to the nasal aperture using inhalation through the nose and the direct application using a spray with an extension nozzle to the pharyngeal mucosa are practical and safe. The nasal application is easier to perform and has advantages in that it may have a more direct effect on the nasal mucosa.

One embodiment of the invention comprises administration of tyloxapol as a solution administered as spray droplets ranging from 50 to 60 μm. In order to maximize deposition to the intermediate and posterior regions of the nose, the spray is squeezed from the bottle in a 30 to 50° arc, and is directed into the nose at an angle of 70 to 80° from the vertical plane of the face. In another embodiment, the solution is applied to the posterior pharynx by means of a spray bottle with an approximately 70 mm extension with a tip which is angled upward toward the roof of the pharynx posterior wall. The volume administered per spray ranges from 0.1 to 0.15 mL, with 1 to 5, preferably 1-3 sprays per nostril and 1 to 5, preferably 2-3 sprays to the posterior pharynx. However, changes in the used concentration of the tyloxapol solution govern the administered volume and number of sprays.

Since the nose, as an organ, has strong filter activity, it might be more effective for treatment of snoring and sleep apnea to use the retrograde, pharyngeal application system. On the other hand, given that numerous subjects have a strong gag reflex, the pharyngeal application might be less practical and useful. The combination of both is therefore most preferred. In this regard, the nasal solution may also be effectively applied to the posterior nasal and velopharynx region by installation through a nasal catheter.

The method of the current invention utilizes, in the most preferred mode, both routes concurrently. Further, when a nasal aerosol or dry powder inhaling system is used instead of the spray pump, deposition and efficacy onto pharynx may be further improved. Numerous such systems are commercially available.

Different doses of the active compound are used for snoring and sleep apnea. The spray is typically applied prior to bedtime, and comprises 1-3 sprays to each nostril and 1-3 sprays to throat of 0.2-20%, preferably 1-10% tyloxapol, for snoring and 3-5 sprays to each nostril and throat of 0.5-20%, preferably 1-15% tyloxapol for sleep apnea. The basic difference between treatment of snoring and sleep apnea is the increased dose used for sleep apnea.

For treatment or prevention of other conditions, as described below, the dosages, regimen and mode of administration is essentially the same. For treatment and prevention of SIDS, the infant is treated with smaller volumes and smaller doses of the active compound. The formulation of the invention provides a different concentration of the active component for different indications and, in case of animal use, such concentration and volume depends on the size of the animal and size of its nasal and pharyngeal area.

V. Methods of Treatment

The invention concerns, in its broadest form, a treatment and prevention of snoring, sleep apnea, sudden infant death syndrome and improvement of sleep patterns and/or nasal breathing following the exercise, or surgery or obstruction of upper airways.

All the above named diseases and conditions, such as snoring, sleep apnea, sudden infant death syndrome and disturbed sleep pattern are basically caused by or resulting from the same pathological mechanism that is caused by or resulting from an occlusion or partial or total collapse of mucosal pharyngeal tissue.

Snoring and sleep apnea are connected as an extension of one to another where snoring is caused by a partial airway occlusion, sleep apnea is caused by a full occlusion of airways. Both snoring and sleep apnea have been associated with disturbed sleep and with ensuing daytime sleepiness and poorer performance.

The symptoms of all these conditions are distinguished by the different outcome following the episodes of snoring or sleep apnea or by the severities of the symptoms. For instance, a person who snores at night is not necessarily very tired in the morning although a subpopulation of snorers with OSAS suffers from daytime fatigue. Snoring is thus more of a social problem than the serious medical condition. Snorers may be classified as suffering from a mild form of sleep disordered breathing. This mild form of sleep disordered breathing is described as obstructive sleep apnea syndrome (OSAS) and the increased nocturnal breathing effort may result in daytime fatigue of the snorer or OSAS patient.

A person suffering from sleep apnea who suffers from severe form of sleep disordered breathing is typically tired and sleepy during the day and may also be prone to an increased blood pressure, electrocardiogram changes, arrhythmia, neurological changes, increased risk of stroke and, typically, exhibits decreased work performance and alertness during the day.

To illustrate how the two conditions are connected, for example, if a person that snores is given a lot of alcohol to drink or anesthesia is administered, such person will convert from a snorer to a person with sleep apnea. Consequently, both these diseases and conditions are treatable basically with the same composition comprising essentially an alkylaryl, such as tyloxapol, as an active ingredient but administered in different doses and regimens. Treatment of these conditions with increasing doses of tyloxapol is logical because of the increased disease severity. Different doses of alkylaryls may also result in longer duration of effect. A higher dose of the alkylaryl for treatment of sleep apnea may be needed than for snoring because the main nuisance of snoring occurs within the first hour of sleep, whereas sleep apnea is a condition which requires a continuous treatment throughout the night.

The method of treatment of the above conditions comprises administration of a formulation comprising one or several alkylaryls in admixture, or one or several alkylaryls in admixture with other surface active compounds and/or pharmaceutically acceptable excipients or additives.

The preferred method of treatment comprises administration of a spray, liquid, solid or aerosol formulation comprising tyloxapol alone or in admixture with one or several other alkylaryls, or tyloxapol in admixture with another surface active compound or compounds and/or in admixture with pharmaceutically acceptable excipients or additives.

The methods for treatment of various conditions listed above differ in the formulation, routes of administration, amount of the active ingredient, dosing and daily dosing regimen.

The method of treatment according to the invention are safe, practical and efficacious.

Extended administration of inhalable tyloxapol is, according to the product description (Tacholiquin SmPC, Bene, Germany, Gebrauchsinformation, 1999), safe and without observable side effects. Tyloxapol, when applied to the airways as an aerosol is especially safe. A similar safety profile is observed upon nasal spray application with only very limited systemic absorption.

The safety of the tyloxapol or other alkylaryl containing compositions is particularly important in this case because of the possible unintended aspiration upon nasal spray or liquid application. Other agents currently proposed for topical treatment of snoring, such as phosphocolinamin, bear the risk of a lipid pneumonia when inhaled (Am. J. Respir. Crit. Care Med., 157:1522-1525 (1998).

A. Treatment and Prevention of Snoring

Snoring is associated with increased pulmonary resistance, reduction in pharyngeal cross-sectional area and reduction in pharyngeal closing pressure, that is the pressure necessary to induce pharyngeal collapse. The combination of large negative intrathoracic pressure generated during snoring increases upper airway resistance, reduces pharyngeal area and increases pharyngeal collapsibility leading to dynamic compression of the upper airway and turbulent airflow through the larynx and oropharynx. Turbulent airflow causes vibration of the soft palate and faucial pillars, resulting in the characteristic snoring noise. Vibrations of the soft palate are always present in conjunction with circumferential narrowing of the velopharyngeal lumen. In addition to palatal vibration, apneic snorers exhibit collapse in the velopharyngeal or hypopharyngeal region.

Currently, the predominant treatment for snoring include positive pressure therapy, oral retainers or jaw protractors and surgery to maintain airway patency, as well as manipulation of sleeping and head position. Pharmacological interventions include administration of various compounds such as nasal dilators, surfactants, including surface active compounds and pulmonary surfactants, lipid based agents and lubricants. However, all remedies currently available have their limitations and few are quite invasive and uncomfortable or may become dangerous.

Lipid based compounds, for example, may lead to lipid pneumonia when aspirated, animal derived surfactants require refrigeration and carry the risk of transmitting prionic disease, synthetic surfactants are very expensive and, of course, surgery is invasive and always carries with it a risk of complications. Moreover and most importantly, none of the above described approaches has been entirely successful to eliminated snoring.

The effective treatment for snoring must be practical, safe, non-invasive and efficacious.

It has now been discovered that a composition comprising alkylaryl, preferably tyloxapol, administered as a spray, liquid, lozenge or aerosol nasally and/or pharyngeally before sleep improves airflow resistance and eliminates or substantially decreases snoring.

The composition of the invention for treatment of snoring is preferably administered to the posterior nose and velopharynx.

To insure that the targeted area is reached, the composition is preferably administered via both the nasal and pharyngeal passageways. The active ingredient acts as a dispersant, mucolytic, antioxidant, anti-inflammatory and anti-scavenger agent in these areas and in airways and its cumulative pharmacological effect results in treatment and prevention of snoring.

Dispersant and mucolytic activity of the active ingredient reduces mucosal adhesive forces and prevents collapse of muscular and epithelial structures in the nose and throat. Anti-inflammatory activity reduces inflammation of epithelium, swelling and epithelial damage. All these effects together result in improvement of airway patency, reduction of airway resistance and general improvement of the clinical symptoms of snoring and sleep apnea.

The method for treatment of snoring has been proven in two clinical trials and in one dose response study to determine the minimal dose needed for treatment of snoring. Exact conditions and results are described in Examples 1 and 2.

In a first study, six subjects with light to severe snoring were treated for a one week period using a treatment regimen of 2 spray squirts per nostril of a composition comprising 1% tyloxapol each night 30 minutes prior to sleep.

Study subjects were light to severe snorers whose snoring disturbed others, had a partner able to observe the effect of treatment, and were otherwise healthy.

Subjects who had nasal infection or cold, nasal abnormality/septum deviation, massive obesity, or used systemic drugs that affect muscular tone were excluded.

Each night, shortly before bedtime, two squirts of tyloxapol (1% per nostril) in 0.2-0.5 ml volume per nostril, were administered. Subjects were observed by their bed partners for one week. Snoring was reported by the bed partner and evaluated on a scale of 0-3, with 0 representing no effect and 3 representing a very good effect (no snoring).

In this study, snoring pattern has improved in all six subjects, with an average repeated score of 2, representing a good effect with no or only light snoring. Nasal breathing improved in all six subjects, with an average score of 2.2. The mucolytic effect was also described in all six subjects, with an average score of 1.3.

A second controlled study on effect of tyloxapol on snoring is described in greater detail in Example 1.

In this study, 12 subjects who suffered from moderate to severe snoring were investigated in a two period study. Visual analog scale (VAS) and apnea/hypopnea (AHI) were used to assess the severity of snoring.

At the end of the study, 92% (11 out of 12) of treated subjects had larger than 50% improvement in snoring periods compared to nights without treatment.

Additionally, in order to determine the lowest possible dose of tyloxapol for treatment of snoring, the dose response study was performed. The dose ranging study compared three different treatment arms in a group of 6 snoring individuals (N=6): untreated treated with 0.1% (1 mg/ml) and the third group was treated with 0.55% (5.5 mg/ml) of tyloxapol. Untreated group and group treated with 0.1% tyloxapol were not significantly different on a visual analog scale (VAS) although some decrease in VAS was noted for group treated with 0.1%. Treatment with 0.55% tyloxapol nasal spray resulted in significantly decreased VAS and was also clinically proven to decrease or eliminate snoring.

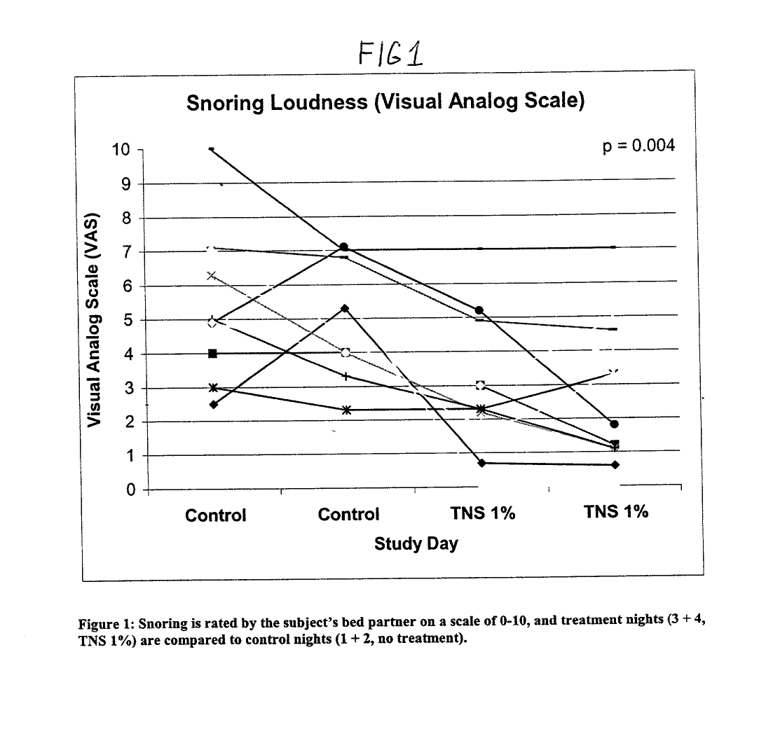

Results of the second study are illustrated in FIG. 1.

FIG. 1 is a graph showing values of snoring loudness, where snoring was rated by the snoring subject's bed partner on a scale of 0-10 and expressed on the visual analog scale. On treatment nights (nights 3 and 4), subjects were given 1% tyloxapol nasal spray approximately 30 minutes before going to bed. Results observed on nights 3 and 4 are compared to control nights 1 and 2 where there was no treatment given.

As seen from FIG. 1, all subjects but one have shown significant improvement in snoring pattern when compared to the control nights. These results clearly show that treatment with 1% tyloxapol prior to sleep greatly decreases or eliminates snoring altogether.

Consequently, the minimal dose of alkylaryl for treatment of snoring, and tyloxapol especially, is at least 0.2%, that is 2 mg/ml of the administered dose, preferably about 1% maximal daily dose administered to the snoring subject is 3 grams.

In practice, snoring subject is treated daily before going to bed, preferably within 10-30 minutes before falling asleep, with a composition comprising 0.2-20% of alkylaryl of choice, preferably with a composition comprising 1-10%, most preferably 1% tyloxapol. The treatment may be repeated every 2 hours or as needed, but will typically substantially decrease or eliminate snoring for at least 4 hours and one treatment before sleep may be sufficient to prevent severe snoring throughout the night.

B. Method for Treatment of Sleep Apnea in Humans

Sleep apnea is among the most troublesome sleep impairments. It is believed that as many as 2-4% of all adults suffer from sleep apnea. Between 7 and 18 million people in the United States suffer from a mild form of sleep disordered breathing, and between 1.8 and 4 million may suffer from a more severe form of this condition (European Psych., 10:109s-113s (1995)).

Sleep apnea has been associated with increased daytime sleepiness and decreased work performance, with increased blood pressure, ECG changes, arrhythmia, neurologic changes, and increased risk for stroke.

The sleep apnea syndrome is characterized by repetitive episodes of upper airway obstruction that occur during sleep. Sleep apnea episodes are defined as cessation of airflow, i.e., complete collapse for more than 10 seconds, occurring usually more than 20 times per hour during sleep, causing measurable blood deoxygenation. Sleep apnea can be obstructive with upper airway blockage despite airflow drive, central with decreased respiratory center output or mixed of those two.

Upper airway narrowing leads to obstruction during sleep. The apneic period lasts from 10 seconds up to 2 minutes. Repeated nocturnal obstruction may cause a repeating cycle of sleep, obstructive choking and arousal with gasping. Daytime drowsiness follows.

An apnea is defined as complete airflow cessation and is accompanied with a blood oxygen drop of 3%. The airflow reduced to below 70% of normal airflow and blood oxygen drop of at least 3%, results in hypopnea. The sum of apneas and hypopneas per hour are termed the apnea-hypopnea index (AHI) or respiratory distress index (RDI). These two are identical indices, and used in clinical evaluation of severity of sleep apnea.

Pathophysiologically, sleep apnea is characterized by recurrent closure of the pharyngeal airway during sleep. Upper airway patency is influenced by muscle activity, anatomical features, vasomotor tone, mucosal adhesive forces, and inflammation.

Currently, as already described above, the predominant treatments for sleep apnea include positive pressure therapy, use of oral apparatuses, and surgical removal of accessory tissue to maintain airway patency. Conservative treatments include weight loss, reduction of alcohol consumption, and avoidance of medications that influence muscular tone. Pharmacologic treatments for sleep apnea typically include the systemic application of various therapeutic agents or nasal application of surfactants and tissue lubricating agents.

Treatments used or proposed for sleep apnea described above have their limitations and insofar none of these treatments result in a well tolerated and efficient treatment of sleep apnea. A successful and efficient treatment for sleep apnea should be noninvasive, safe, practical and have a demonstrable efficacy.

Following the finding that nasal and/or pharyngeal application of an alkylaryl compound reduces airflow resistance, improves airway patency and decreases or eliminates snoring, the effect of tyloxapol on sleep apnea has been investigated.

Tyloxapol is known to act as a dispersant, a mucolytic, an antioxidant, an anti-inflammatory agent, and a free radical scavenger. These activities of tyloxapol contribute to its usefulness as anti-snoring and anti-apnea agent. Dispersant and mucolytic action of tyloxapol reduces mucosal adhesive forces and helps prevent the collapse of muscular and epithelial structures in the nose and throat. Furthermore, the anti-inflammatory activity of tyloxapol protects the epithelium from swelling and damage. It has now been discovered that as a nasal and/or pharyngeal spray, administration of tyloxapol or another alkylaryl improves airway patency by reducing inspiratory airway resistance and the inspiratory effort in sleep apnea. This treatment has been found to improve the clinical symptoms of sleep apnea, measured as improved sleep efficiency, deeper sleep (more stage 3 and 4 NREM and more REM sleep), and reduction of arousals (ArI) and apneic and hypopneic events (AHI/RDI).

A study performed for determination of therapeutic utility of tyloxapol on sleep apnea was an open clinical study with 1% tyloxapol nasal solution (TNS) in patients with moderate to severe sleep apnea undergoing evaluation in a sleep laboratory. Tyloxapol composition as used for one month or longer and no adverse reaction were observed during this time.

An objective of this study was to determine efficacy of tyloxapol containing composition in the treatment of sleep apnea and to determine the safety and efficacy of the tyloxapol formulation for long term treatment.

Polysomnography was used to assess efficacy. Apneic and hypopneic episodes per hour were measured using the Apnea Hypopnea Index (AHI). The effects on the quality of sleep were assessed by measurement of REM sleep as a percentage of total sleep (REM %), percentage of non-REM sleep (NREM %), percentage of stage 1, 2, 3 or 4 (NREM % Stage 1 or 2 and NREM % Stage 3 or 4), arousal index (ArI), and sleep efficiency (SE).

The effect on oxygen desaturation associated sleep events was assessed by measuring oxygen saturation (O₂), the number of desaturations, and their magnitude (D Sat O₂).

Polysomnography, as used in this study, was an attended, full night recording of airflow, arterial oxygen saturation, respiratory effort, electrocardiogram (ECG), electro-oculogram (EOG), electro-encephalogram (EEG) including sleep staging, muscle activity, electromyogram (EMG), and variable parameters such as snoring noise recordings.

The AHI, a measure of apneic and hypopneic events per hour, served as a measure of apneic collapse of the upper airways, and decrease of respiratory flow. By continuous recording of arterial oxygen saturation, events associated with desaturations were assessed to help understand the direct impact of breathing cessation on brain and tissues. The quantity of sleep was assessed via measurement of arousal (arousal index, ArI) and EEG determined sleep efficiency (SE) by recording time asleep. The amount of REM sleep (REM %), as well as the amount of non-REM sleep (NREM % Stages 1 or 2 and NREM % stage 3 or 4) serves as an indicator of depth and of the refreshing quality of sleep. In the available literature, the apnea-hypopnea index, (AHI), as well as the different measures of sleep quality and quantity, are widely accepted determinants of the extent and clinical severity of sleep apnea (Amer. Sleep Disorders Association and Sleep Research Society, 20 (6): 423-487 (1997)).

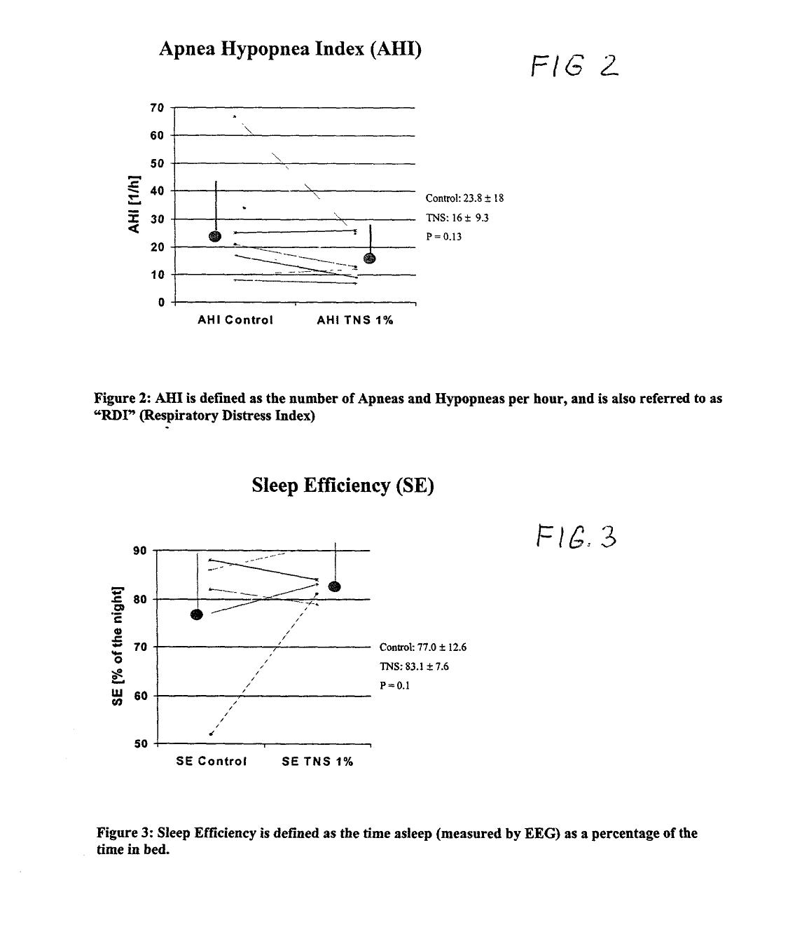

Results of the polysomnography performed on sleep apnea patients are shown in FIGS. 2-4.

FIG. 2 illustrates a decrease in apneic/hypopneic episodes in patients treated with 1% tyloxapol. The number of apneic hypopneic episodes are expressed as apnea hypopnea index (AHI). As seen from FIG. 2, in average there were 23.8 apneic/hypopneic episodes recorded in control patients compared to an average of 16 apneic/hypopneic episodes recorded following treatment with 1% tyloxapol nasal spray.

FIG. 3 illustrates the effect of tyloxapol nasal solution on improvement of sleep efficiency defined as the time asleep as a percentage of the time in bed. This time is measured by electroencephalogram. As seen from FIG. 3, the sleeping time in the patient treated with tyloxapol has increased from the 77% to 83.1% per night.

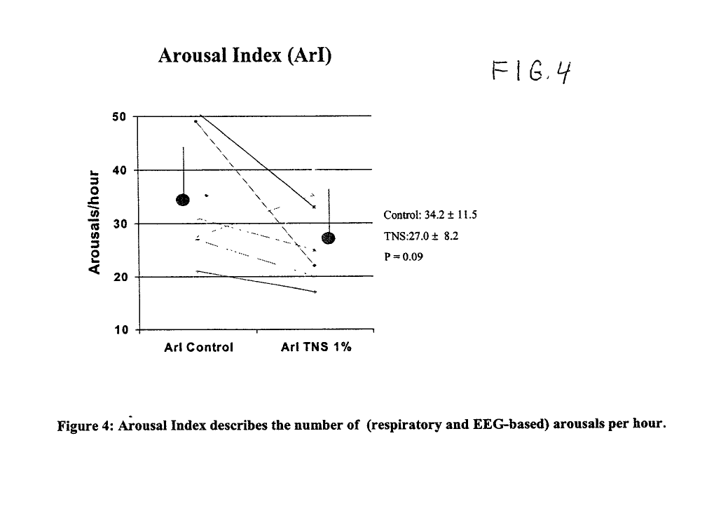

FIG. 4 illustrates decrease in occurrence in number of arousal periods per one hour, as measured by arousal index (ArI). As seen from FIG. 4, where the control arousal index was 34.2 per hour, following treatment with tyloxapol, such treatment resulted in decrease in ArI to 27 arousals/hour.

Results seen in FIGS. 2-4 show that treatment with tyloxapol is effective for treatment of sleep apnea and improvement of sleep pattern.

In practice, the treatment of sleep apnea comprises administering the alkylaryl polyether alcohol polymer, preferably tyloxapol, containing composition which, when applied to the upper airways, eliminating or reducing apneic/hypopneic events to less frequent and less severe episodes.

The minimal dose of alkylaryl, and tyloxapol especially for treatment of sleep apnea, was determined to be at least 0.5%, that is 5 mg/ml of the administered dose, and may be as high as 20% (20 mg/ml), depending on the severity of sleep apnea and on other symptomatic characteristics of a patient, such as for example, obesity, pulmonary obstruction, age and general state of health of the patient, etc.

The apneic patient is treated within 10-60 minutes, preferably within 10-30 minutes before sleep with an alkylaryl containing solution (0.5-20%) by 1-5, preferably 3 spray doses into each nostril and 2-5 squirts pharyngeally. The dose may be repeated each 2-8 hours, as needed throughout the night. The total daily dose should not exceed 3 grams administered in the most concentrated 20% solution.

C. Method for Prevention of Sudden Infant Death Syndrome

A nasal and throat spray application of an alkylaryl polyether alcohol polymer, preferably tyloxapol, is also suitable for prophylaxis and reduction, occurrence and extent of apneic episodes in infants, known as sudden infant death syndrome (SIDS).

SIDS is defined as the sudden death of an infant younger that one year of age that remains unexplained after a thorough case investigation that includes a complete autopsy, examination of the death scene, and review of the clinical history.

Apparent life threatening event (ALTE), also called a near-miss SIDS, is defined as an episode that is characterized by apnea, color change, change in muscle tone, choking or gagging and is frightening to the observer.

Sudden infant death syndrome (SIDS) accounts for the largest number of deaths during the first year of life in developed countries. Among sudden and unexpected infant deaths, 80-82% were diagnosed as SIDS (J. Pediatr., 135(4): 437-443 (1999) and Arch. Dis. Child, 82:98-106 (2000). According to Pediatrics, 104:1229-1246 (1999) making SIDS the third leading cause of infant mortality (8.9%) after congenital anomalies (22%) and short gestation/low birth weight (14%).

The possible causes of SIDS are numerous and, to date, there is no adequate unifying pathological explanation for SIDS, although it is generally believed that the SIDS is caused by dysfunctional reflex mechanisms for awakening and breathing where the apneic episodes of the infant are not ended by awakening, as is the case for the sleep apneic adult.

There is no specific autopsy finding pathognomonic of SIDS and no finding required for the diagnosis. Some common indicators for SIDS, such as petechial hemorrhages found in more than 70-90% of cases and pulmonary edema, are often present and may be substantial. Due to lack of uniform criteria among pathologists, some unexpected infant deaths may be misdiagnosed as pneumonia or other natural causes based on minimal findings at autopsy which are insufficient to explain the infant's sudden death.

J. Pediatr., 128:594-596 (1996) describes certain putative interactions which exist between sleep position, soft bedding and blankets, and impaired cardiorespiratory control, especially impaired ventilatory and arousal responsiveness. Face down sleeping, which occasionally occurs in healthy full-term infants who are sleeping prone, may result in transient episodes of airway obstruction and asphyxia, however, these healthy infants are typically able to awake on their own. On the other hand, infants with insufficient arousal responsiveness to asphyxia may not awake and are thus at risk for fatal asphyxia, i.e., sudden death.

There also appear to be interactions between sleep position and impaired thermoregulation (Early Human Dev., 43:109-116 (1995). Face down sleeping can cause clinically significant thermal stress, which may further compromise infants with deficient cardiorespiratory control or autonomic control, especially those with genetic or acquired susceptibility to impaired thermoregulation.

Additionally, the roles of environmental risk factors and pathophysiological abnormalities for SIDS have been established as causation for SIDS. Environmental factors include prone and side sleep positions, soft bedding and blankets, maternal cigarette smoking and drug intake, overheated sleeping quarters and upper respiratory infections. Pathophysiological abnormalities include impaired ventilatory and arousal responses to hypoxia and hypercarbia, autonomic dysregulation, immune dysfunction, and thermoregulation (Am. J. Respir. Crit. Care Med., 164; 346-35, (2001)). Similarly to sleep apneic adults, children of patients with obstructive sleep apnea syndrome appear to have inherited subtle defects that reduce their ability to compensate for increased loads due to, for example, obesity and to maintain upper airway patency during sleep (Am. J. Respir. Crit. Care Med., 155 (5):1602-1608 (1997).

Further, familial sleep-disordered breathing may be based partly on a familial abnormality in ventilatory control associated with blunting of the hypoxic ventilatory response. In the study of children of index adults with obstructive sleep apnea syndrome and first and second degree relatives, a trend toward higher incidence of SIDS/ALTE (10.8% versus 3.2%, p=0.11) in the sleep apnea syndrome index families was found. All 10 SIDS/ALTE events occurred in families where at least one adult was diagnosed or suffered with obstructive sleep apnea syndrome.

These studies show that ALTE/SIDS and sleep apnea share some of the same pathomechanisms, such as for example the occlusion of upper airway. Due to the immaturity of the infant's brain, the outcome of the sleep apnea episode may result in more severe (ALTE) or fatal (SIDS) consequences. When the apneic episode of the infant is not ended by normal, healthy awakening, as is the case for the adult sleep apneic, either ALTE or SIDS result.

It has now been discovered that preventive administration of a nasal and throat spray or liquid solution comprising alkylaryl polyether alcohol polymer, preferably tyloxapol, to a predisposed infant reduces the occurrence and extent of apneic episodes which is an apparent life threatening event in these infants. Such administration of tyloxapol or another alkylaryl thus serves as a prophylaxis for sudden infant death syndrome.

The tyloxapol or another alkylaryl polyether alcohol polymer comprising spray or liquid solution in from about 0.01 to about 0.5% (0.1 to 5 mg/ml) is a safe topical treatment for infants. A tyloxapol nasal/pharyngeal spray is a safe prophylactic to occurrence of ALTE and/or SIDS. The mode of action of tyloxapol for prevention of SIDS encompasses, among other effects, also the improvement of upper airway patency by reducing opening pressure, as well as the described anti-inflammatory effect of the compounds.

In practice of the invention, the infant in need of such treatment, that is the infant either predisposed to the ALTE or SIDS because of genetic, pathophysiological or environmental conditions, is treated with a composition comprising from about 0.01 to about 0.5% of tyloxapol or another alkylaryl within 5 to 60, preferably within 10-20, minutes before sleep by administering the spray as a squirt of about 0.1 to about 0.5 ml or about 1-10, preferably 3-5 drops either to the infant's nose, pharynx or both.

In infants suffering from bronchial infection or inflammation the composition is administered not only before sleep but also during the day to improve the infant's breathing. The composition is administered once, twice or more times per day as needed. The recommended total dose which should be administered per day should typically not exceed 1 gram per day. In any case, the total dose for treatment or prevention of SIDS and/or ALTE contains a lower concentrations of tyloxapol than the one used for the treatment of sleep apnea and snoring.

D. Method for Improvement of Upper Airway Patency

The method and composition of the invention is also suitable for improvement of upper airway patency and improved nasal breathing resulting, by extension, in improved physical performance during exercise, such as flying, hiking, mountain climbing, or diving and in improvement of sleep pattern, reduction of daytime fatigue and increased alertness.

The collapsible upper airways and related structures, such as sinuses, Eustachian tubes, and middle ears, are subject to compressive forces during activities like strenuous exercise, diving and flying. Dysbarism, also known as nose-ear distress syndrome is commonly caused by dysfunction of the Eustachian tube or nasal congestion. This dysfunction is a common cause of ear, nose and throat (ENT) infections, headache and other pathologies. Similarly, the barotrauma occurring during and after flying is caused by compression and decompression of upper airways.

In general, the effect of topical tyloxapol application on upper airway patency is observed within seconds after the administration of the alkylaryl. The duration of such effect depends on the dose and on the concentration applied. Investigated snoring patients that have been using the tyloxapol spray before going to bed reported a nocturnal duration of such effect of about 3 to 4 hours.

When applying tyloxapol prior to exercise, the duration of effect is shorter because the metabolism is faster during exercise and the washout of the alkylaryl from the epithelial surface during exercise is also faster than during the sleep at night. Also, the nasal patency is more critical for exercise and performance typically performed in vertical rather than horizontal position than for snoring and sleep apnea where the upper airway collapse occurs in the velopharyngeal region and not in the nasal area.

In practice, for improvement of nasal breathing during the physical activity, nasal spray, nasal drops or lozenges are administered prior and/or during the activity. Nasal instillation techniques for those indications are more effective than throat sprays, however, lozenge use is more convenient and practical in these instances.

Pretreatment before exercise and endurance activities as well as activities with short term respiratory and cardiovascular loads comprises administration of a nasal composition, preferably a nasal spray comprising 1 to 20%, preferably 5-20%, tyloxapol or another alkylaryl polyether alcohol polymer, in 2-3 squirts of 0.1 to 0.2 ml each to each nostril applied within 10-15 minutes prior to the start of the exercise. The nasal spray may be re-applied every 1-2 hours during the activity, or even earlier, when the effect has weakened due to a washout effect. Lozenge may be used to substitute for the spray or supplement it.

The above described pretreatment is suitable for use in mammals in general, and is both safe and effective for humans as well as for animals.

Pretreatment of and treatment during and after flying, climbing, mountain hiking and diving is intended to improve the patency of the nose and middle ear, especially the eustachian tube, connecting the two areas as these activities are associated with increased or decreased pressure. Most of the pressure problems and complications in diving and flying stem from occlusion and negative pressure in the Eustachian tube area. The pretreatment of these activities consists of administering an alkylaryl comprising composition, preferably a nasal spray comprising 1 to 20%, more preferably 5-15% tyloxapol administered as 1-3 squirts of 0.1 to 0.2 ml each to each nostril, applied within 10-15 minutes prior to flying, hiking, climbing, diving or before takeoff of a plane. The composition may be re-applied every 1-2 hours during the flight, or sooner during the strenuous exercise in thinner air during high altitude climbing or hiking or at any time when the effect has weakened due to a washout effect.

Additionally, further administration may be appropriate after diving, in order to facilitate ventilation of the Eustachian tube, thereby preventing infections and entrapment of water.

The composition of the invention may also be used for easing symptoms of upper airway infections, disease and irritation, such as stuffed or runny nose or minor colds. The composition of this invention promotes moistening of the epithelial surfaces of the nose and upper airways and reduces friction and inflammation and thus decreases symptoms connected with colds, viral infections as well as Sjøgren's disease where the patient suffers from dry epithelia due to an autoimmune disease.

Many ear, nose, and throat (ENT) problems will also benefit from the above mentioned treatment. Examples of these diseases suitable for treatment with a composition of the invention are nasal allergies, rhinitis sicca, and sinusitis. Septum deviations and other anatomical abnormalities often lead to impaired nasal breathing, perpetuated by epithelial swelling and inflammation. Also, the patency of the Eustachian tube (and the middle ear), which is impaired in children, may show benefit from the alkylaryl polyether alcohol polymer treatment. ENT surgery and the treatment before and after an operation and the anatomically impaired nasal passages as, for example, in a case of or after nasal trauma, septum deviations, etc, are another indication for treatment according to the invention.

There are other medical indications where the compositions of the invention improves a health and conditions of the patients. In intensive care, for example, nasal or oropharyngeal intubation for ventilation, including tracheotomy, leads to chronic damage of upper airways, their increased collapsibility and impairment of breathing. A treatment according to the invention instituted concurrently with intubation/tracheotomy eases such problems and is beneficial in that the tolerance of longer term intubation may be improved by spray application of alkylaryl polyether alcohol polymers to the epithelium.

For these indications, a tyloxapol nasal spray (1 to 15% tyloxapol or alkylaryl polyether alcohol polymer) is administered in a dose of 3 sprays of 0.1 to 0.2 ml each to each nostril, applied several times per day up to a maximum dose of 3 grams of tyloxapol or other alkylaryl per day is reached. The composition may be applied by nasal or pharyngeal spray, by direct nasal instillation of nasal drops, by nasal inhalation of dry powder as well as using oral lozenges.

Further, alkylaryl polyether alcohol polymer spray treatment may have a synergistic effect in combination with a topical, nasal drug application of antibiotics, anti-inflammatory drugs or decongestants.

Additionally, the current formulation may be advantageously used by performance artist such as singers, vocalists and actors where the upper airway patency is constantly being challenged. The stress on the upper airways that is caused by extended and continuous performance sometimes leads to chronic inflammation of the throat and upper airways.

Continuous and chronic treatment with the composition of the invention provides and assures increased patency in the pharynx and also results in reducing friction and thereby, inflammation in the larynx and surrounding tissues. The preferred mode of administration is a throat spray, a combination of nasal and throat spray, or use of oral lozenges comprising from about 0.5 to 15% tyloxapol or another alkylaryl polyether alcohol polymer. The composition is administered prior to practice or performance, and repeated when needed.

E. Animal Application

The animal equivalents to human conditions benefitting from or requiring treatment according to the current invention are species-specific impairment of nose breathing, and obligate mouth-breathing in certain breeds of dogs, cats, horses and other mammals.

Nasal breathing and openness of the nasal passageways are crucial for ventilation during strenuous exercise/physical activity not only in humans but also in other mammals. Consequently, mammals with anatomically or functionally narrowed nasal passageways or those exposed to abnormally demanding physical activity and performance, such as racing horses or dogs are likely to benefit from such treatment. Specifically, one of the quality criteria for racehorses is the patency and width of their nostrils allowing them to inhale large amount of air. Thus, both race horses as well as performance dogs benefit from an alkylaryl containing nasal spray treatment that results in improved nasal breathing.

In mammals, the conducting upper airways provide for the passage of air to the lungs, just as in humans. Clinical problems in horses, dogs, cats and other mammals can be manifested in stertorous, sonorous breathing, dyspnea, and in chronic bouts of inspiratory stridor and cough. Concurrent disease of the lower respiratory system as well as other organ systems (cardiovascular, nervous, endocrine) contribute to the pathology.

Many upper airway problems in animals, especially those with congenital conditions, need surgical and/or pharmacological intervention. Historically, a variety of drugs, including decongestants, cough suppressants, bronchodilators, glucocorticoids, and antibiotics have been advocated. Even systemic aspirin and digitalis have been proposed.

Impaired nasal breathing and heaves, septum deviation, and choanal deviations are common problems in horses and foals and the relationship between airway patency and an animal's quality of life and performance has been described. The same applies to many breeds of dogs especially in the case of brachiocephalic breeds, such as Pekinese, Boxers, Boston terriers, and the Mops and King Charles Spaniels. Guinea pigs are also found to have such problems. Among cats, it is mainly the Persian variety that is affected.

These breeds have impaired nasal breathing due to congenital and anatomical variations, like a rudimentary nose, and sometimes even missing tear ducts. In pigs and cows, these symptoms are also not uncommon, especially in combination with viral infections or rhinitis atrophicans, and present a therapeutic dilemma. They also occur in sheep, although less frequently.

The current invention provides a formulation comprised of tyloxapol or another alkylaryl polyether alcohol polymer in veterinary composition for treatment of animal respiration problems. The nasal and/or pharyngeal spray application of an alkylaryl polyether alcohol polymer, particularly tyloxapol, reduces clinical symptoms, improves airway patency, and improves the animal's quality of life or performance.

In practice, an animal in need of such treatment is administered a nasal spray or solution comprising 1-15% tyloxapol or another alkylaryl administered in a volume which is sufficient to coat the animal's nasal cavity. The administered volume thus depends on the size of the animal and on the severity of affliction. The maximal daily dose administered to animals, particularly to large animals such as horses, may exceed 10 g/day.

### UTILITY

The invention is useful for treatment of snoring, sleep apnea, SIDS and for improvement of nasal breathing. The method for treatment is safe, practical and noninvasive. The active components of the therapeutic composition useful for treatment are non-toxic and safe in far larger concentrations that those needed for treatment of snoring, sleep apnea, SIDS and improvement of nasal breathing according to the current invention.

Further advantage of the current invention is the fact that an alkylaryl polyether alcohol polymer such as tyloxapol is substantially less costly than lung surfactants, and may even be accessible over the counter as opposed to prescription drugs.

The invention is described in illustrative examples. These examples should not be interpreted as in any way limiting the scope of the invention.

### EXAMPLE 1

### Effect of Tyloxapol on Snoring

This example describes a clinical study for determination of effect of tyloxapol on snoring.

The clinical study was an open label, two period study of 1% tyloxapol nasal spray (TNS) in 12 patients (mean age 60.7 years; 10 males) with moderate to severe snoring. Three of the 12 patients were also diagnosed with sleep apnea.

The study design consisted of two nights without treatment followed by two nights of treatment with three squirts (0.15 ml each) of a 1% TNS solution per nostril and three squirts pharyngeally (total 1.26 ml, e.e., 12.6 mg total), administered no later than 30 minutes before bedtime. Efficacy was evaluated with a visual analog scale (VAS) assessing the severity of snoring, and the Bedbugg® Home Monitoring Device. Bedbugg® is a portable recording device for assessment of sleep lab parameters including the apnea/hypopnea index (AHI).

The administered formulation contained 10 mg of tyloxapol, 50 mg glycerol, 20 mg sodium in bicarbonate per 1 ml.

Inclusion criteria for study subjects included light to severe snoring that disturbed others; constant partner for the observation of the effect; and good health and no interfering medication. Patients with nasal infection/cold, nasal abnormality/septum deviation, massive obesity, and/or use of systemic drugs that affect muscular tone were excluded.

At the end of the study, 11 out of 12 patients (92%) had shown improvements in their VAS scores (p=0.004). On average, the VAS scores were more than 50% lower on treatment nights, with averages of 3.1 and 2.8, compared to 5.4 and 5.1 on nights without treatment.

The results of the VAS analysis clearly demonstrate the efficacy of TNS 1% in the reduction of snoring. Among the three apneic patients, two had reductions in their AHI scores, suggesting reductions in apneic events.

### EXAMPLE 2

### Dose-Response Study

This example describes a dose response study to determine the lowest possible dose of tyloxapol effective on snoring.

Six patients were included in this study. The study lasted for six nights. Administration regimen included two nights sleep without drug, two nights treatment with 0.1% tyloxapol nasal solution (TNS) and two nights treatment with 0.55%, delivered as three squirts per nostril.

Tyloxapol composition consisted of 1 mg/ml or 5.5 mg/ml of tyloxapol, 50 mg of glycerol and 20 mg of sodium bicarbonate of sterile water.

At control nights 1 and 2, subjects received no treatment.

TABLE 1

| | | Visual Analog Scale |
|---|---|---|
| | | (bedpartner) |
| No treatment, control | | 6.1 ± 2.3 |
| 0.1% | TNS | 4.8 ± 2.4 |
| 0.55% | TNS | 2.3 ± 2.0* |

*p = 0.01, different from control, paired t-test

In comparing the groups, a clear relationship between tyloxapol dose and snoring effect could be seen. A minimum dose of 0.55% Tyloxapol (5.5 mg/ml; total dose of 4.62 mg) is required to demonstrate a significant effect on snoring.

VAS scores were lower on nights on tyloxapol 0.1% (VAS 4.8±2.4), but did not differ significantly from control nights. However, on nights using tyloxapol 0.55% nasal spray, significantly lower VAS scores (VAS 2.3±2.0) were recorded than on control nights (VAS 6.1±2.3, p=0.013).

### EXAMPLE 3

### Effect of Tyloxapol On Sleep Apnea

This example describes the effect of tyloxapol on sleep apnea.

The study was performed at a hospital Kloster Grafschaft at Schmallenberg-Grafschaft, Germany under supervision of Dr. B. Schoenhofer.

In this open case series, 1% TNS was administered to 10 patients (8 male, mean age 50.2±10.6 years) with sleep apnea undergoing diagnostic evaluation in a sleep laboratory.

Sleep apnea was defined as having an apnea/hypopnea index (AHI) of 10 and above. Patients (outpatients) were treated with 5 sprays (0.15 ml each; i.e., 22.5 mg total) of a 1% tyloxapol solution to each nostril and 5 sprays in the throat. Therapy was continued for one month (outpatient). Polysomnography study data from the control night (the night before) was used as a control.

The major endpoints included determination of AHI, arousal index (EEG change in depth of sleep), sleep efficiency (EEG % time sleeping), and % REM sleep.

The baseline average AHI (events/hr±SD) was 23.8±18 and decreased to 16.4±9.3 (p=0.13) on TNS. The baseline average arousal index (events/night±SD) was 34.2±11.5 and decreased to 27.0±8.2 (p=0.09) on TNS. The baseline average sleep efficiency (%±SD) was 77.0±12.6 and improved to 83.1±7.6 (p=0.1) on TNS. The baseline average REM sleep (% night SD) was 11.0±7.8 and improved to 15.8±5.2 (p=0.11) on TNS. The amount of deep sleep (NREM stages 3 and 4) increased from 13.3±7.3 to 17.0±8.5% and the lighter sleep stages 1 and 2 decreased from 75.8±9.6 to 67.7±10.3%. No adverse effects were reported, and therapy was continued for one month (outpatient). After one month, 7 out of 10 reported reduction of snoring and were still using the medication.

The data show apneas reduced by more than 30%, indicating positive results with a low dose of 1% of tyloxapol. REM sleep % increased by more than 40%, indicating improvement in quality of sleep. The increase in REM sleep and deep sleep is considered as increasing the quality of sleep and is associated with improved memory and overall mental performance.

What is claimed:

1. A method for treatment or prevention of snoring, sleep apnea or sudden infant death syndrome and for improvement of nasal breathing in mammals, said method comprising a step of administering to a subject in need thereof a liquid or a solid composition comprising of from about 0.01 to about 20% an alkylaryl polyether alcohol polymer nasally or pharyngeally.

2. The method of claim 1, wherein the alkylaryl polyether alcohol polymer comprising composition is applied from antegrade and from retrograde.

3. The method of claim 1, wherein the alkylaryl polyether alcohol polymer is tyloxapol.

4. The method of claim 3 wherein the composition is liquid composition applied as a nasal or pharyngeal spray, as a nasal solution, as a dry powder, as a lozenge or as a nasal aerosol.

5. The method of claim 4 useful for treatment and prevention of snoring in humans comprising administration of the composition comprising from about 0.2 to about 20% of tyloxapol.

6. The method of claim 5 wherein the composition comprises from about 1 to about 10% of tyloxapol.

7. The method of claim 6 wherein the composition is the nasal or pharyngeal spray.

8. The method of claim 7 wherein the composition comprises about 1% of tyloxapol.

9. The method of claim 4 useful for treatment and prevention of sleep apnea in humans comprising administration of the composition comprising from about 0.5 to about 20% of tyloxapol.

10. The method of claim 9 wherein the composition comprises from about 5 to about 15% of tyloxapol.

11. The method of claim 10 wherein the composition is the nasal or pharyngeal spray.

12. The method of claim 11 wherein the composition comprises about 5% of tyloxapol.

13. The method of claim 4 useful for treatment and prevention of sudden infant death syndrome in infants comprising administration of the composition comprising from about 0.01 to about 5% of tyloxapol.

14. The method of claim 13 wherein the composition comprises from about 0.1 to about 2% of tyloxapol.

15. The method of claim 14 wherein the composition is the nasal spray or nasal solution.

16. The method of claim 15 wherein the composition comprises about 0.1% of tyloxapol administered to an infant before sleep as 1-3 drops.

17. The method of claim 4 useful for improvement of nasal breathing in humans comprising administration of the composition comprising from about 0.2 to about 20% of tyloxapol.

18. The method of claim 17 useful for improvement of nasal breathing during physical activity or for improvement of nasal breathing impaired due to a disease, infection or surgery by administering to a subject in need of such treatment the composition comprising from about 0.5 to about 10% of tyloxapol.

19. The method of claim 18 wherein the physical activity is diving, mountain hiking, high altitude mountain climbing or flying and wherein the composition is the nasal or pharyngeal spray or lozenge.

20. The method of claim 19 wherein the composition comprises about 1% of tyloxapol formulated as nasal drops, spray or lozenge.

21. The method of claim 4 useful for improvement of nasal breathing in animals.

22. The method of claim 21 wherein the treatment for improvement of nasal breathing in animals comprises administration of the nasal spray composition comprising from about 0.2 to about 20% of tyloxapol.

23. The method of claim 22 wherein the composition comprises from about 5 to about 15% of tyloxapol.

24. The method of claim 1 wherein the composition comprises from about 1% to about 10% of the alkylaryl polyether alcohol polymer, 50 mg glycerol, and 20 mg sodium bicarbonate in aqueous solution or normal or diluted saline.

25. A device for administration of a nasal or pharyngeal composition comprising from about 0.01 to 20% of alkylaryl polyether alcohol polymer suitable for treatment and prevention of snoring, sleep apnea, sudden infant death syndrome or for improvement of nasal breathing.

26. The device of claim 25 wherein the device is a spray container, spray vial, spray pump, atomizer, nebulizer, aerosolizer, dry powder inhaler, humidifier or a mask.

27. The device of claim 26 wherein the mask is a nasal mask suitable for application of continuous positive airway pressure.

28. The device of claim 26, wherein the is a spray container suitable for administration of the composition to the nasal or upper pharyngeal mucosa using an extension nozzle.

29. The method of claim 4 wherein the composition is a dry powder and the device is the dry powder inhaler.

30. A nasal or pharyngeal composition for treatment or prevention of snoring, sleep apnea, sudden infant death syndrome and improvement of nasal breathing in mammals, comprising form about 0.1 mg to about 200 mg of an alkylaryl polyether alcohol polymer alone, in combination with another alkylaryl polyether alcohol polymer, or in admixture with a pharmaceutically acceptable excipient or additives.

31. The composition of claim 30, wherein the alkylaryl polyether alcohol polymer is tyloxapol and the composition is formulated as a nasal spray, nasal solution, nasal drops, lozenge or dry powder.

32. The composition of claim 31 wherein tyloxapol is in concentration from about 1 mg to about 100 dissolved in normal or diluted saline.

33. The composition of claim 32 wherein diluted saline is quarter normal or half normal saline.

34. The composition of claim 31, formulated as a dry powder or lozenge.

35. The composition of claim 34 formulated as the dry powder having a particle size between 5 and 100 microns.

36. The composition of claim 34 formulated as the lozenge.
